(19) United States
(12) Patent Application Publication
(10) Pub. No.: US 2014/0142055 A1
(43) Pub. Date: May 22, 2014

(54) METHODS OF TREATING RECURRENT MEIBOMIAN GLANDS DISORDER AND THEREBY DECREASING THE FREQUENCY OF RECURRENCE

(71) Applicants: Kamran Hosseini, Hayward, CA (US); Lyle M. Bowman, Pleasanton, CA (US)

(72) Inventors: Kamran Hosseini, Hayward, CA (US); Lyle M. Bowman, Pleasanton, CA (US)

(73) Assignee: INSITE VISION CORPORATION, Alameda, CA (US)

(21) Appl. No.: 14/233,152

(22) PCT Filed: Jun. 29, 2012
(86) PCT No.: PCT/US2012/044949

§ 371 Date: Jan. 15, 2014

Related U.S. Application Data
(60) Provisional application No. 61/502,490, filed on Jun. 29, 2011.

(51) Int. Cl. A61K 31/7052; A61K 31/573

(52) U.S. Cl. CPC A61K 31/7052; A61K 31/573
USPC 514/29

(57) ABSTRACT

A method of treating a recurring meibomian gland disorder in a patient includes administering to a patient suffering from recurring meibomian gland disorder a composition comprising a therapeutically effective amount of an azalide antibiotic and a glucocorticoid. The method may thereby reduce the frequency of recurrence of the meibomian gland disorder.

### TECHNICAL FIELD

This application relates generally to methods for treating ocular diseases and, more specifically to methods for treating recurrent meibomian gland disorders and thereby decreasing the frequency of recurrence.

### BACKGROUND

Meibomian glands, also known as tarsal glands, are meibum secreting sebaceous glands located at the eyelid rim. These glands number about 50 in the upper eyelid and about 25 in the lower. Dysfunctional meibomian glands cause ocular disorders such as dry eyes and blepharitis, as the lack of meibum causes dry skin to shed from the eyelid, increasing the chances of ocular infection. When meibomian glands are inflamed, (a condition known as meibomitis, meibomian gland dysfunction, or posterior blepharitis), they become obstructed by thick secretions. As a result of obstruction, the glands may swell; the resulting swelling is termed a chalazion. Obstructed meibomian glands may also be degraded by bacterial lipases, resulting in the formation of free fatty acids, which irritate the eyes and sometimes cause punctate keratopathy.

A common ocular disorder caused by dysfunctional meibomian glands is nonbacterial blepharitis. Blepharitis is characterized by inflammation of the eyelid margins. Blepharitis may cause redness of the eyes, and itching and irritation of the eyelids in one or both eyes. Blepharitis can appear along with various dermatological conditions including, for example, seborrheic dermatitis, rosacea, and eczema.

Blepharitis occurs in two main forms. The first type, anterior blepharitis, affects the outside front of the eyelid near the eyelashes. The two most common causes of anterior blepharitis are Staphylococcus bacterial infection and seborrheic dermatitis. The second form, posterior blepharitis, affects the inner eyelid and can be caused by problems with the meibomian glands. Two skin disorders that commonly cause this form of blepharitis are acne rosacea, which leads to red and inflamed skin, and seborrheic dermatitis.

Blepharitis has a strong tendency to recur and if left untreated can lead to conjunctivitis and the eyelids can ulcerate in some circumstances. It is most commonly treated, although not cured, via a thorough hygiene regimen that helps remove crusts and some bacterial organisms.

Another common ocular disorder due to dysfunctional meibomian glands, as discussed above, is the formation of a chalazion. Topical antibiotic eye drops or ointment may be used for the initial acute infection, but are otherwise of little value in treating a chalazion. If they continue to enlarge or fail to settle within a few months, smaller lesions may be injected with a corticosteroid and larger lesions

Chalazia may be surgically removed. The excision of larger chalazia may result in visible hematoma around the lid, which will wear off within three or four days, whereas the swelling may persist for longer.

Thus, there exists a need for improved treatments for decreasing the frequency of meibomian gland disorders. The present disclosure satisfies this need and provides related advantages as well.

### SUMMARY

In one aspect there is provided use of a composition comprising a therapeutically effective amount of an azalide antibiotic and a glucocorticoid for the treatment of recurring meibomian gland disorder in a patient, wherein said composition reduces the frequency of recurrence of the meibomian gland disorder.

In another aspect, provided is the use of an azalide antibiotic and a glucocorticoid for the manufacture of a composition for the treatment of recurring meibomian gland disorder in a patient, wherein said composition reduces the frequency of recurrence of the meibomian gland disorder.

In another aspect there is provided a method of treating a recurring meibomian gland disorder in a patient, comprising administering to a patient suffering from recurrent meibomian gland disorder a therapeutically effective amount of an azalide antibiotic and a glucocorticoid, thereby reducing the frequency of recurrence of the meibomian gland disorder. In certain embodiments, the patient suffering from recurrent meibomian gland disorder is treated with a combination of azithromycin and dexamethasone. In other embodiments, the initial occurrence of meibomian gland disorder in the patient was treated with a therapy other than a combination of azithromycin and dexamethasone.

The method may include a step of administering the azalide antibiotic and glucocorticoid in a slow release ophthalmic carrier. This carrier may be administered to the eye or eyes of the patient in drop from or via a depot. The previous occurrence or occurrences of meibomian gland disorder in the method was treated with a therapy other than a combination of azithromycin and a glucocorticoid. The glucocorticoid may be dexamethasone and may be present at about 0.1% by weight. An example of an azalide antibiotic used in the method is azithromycin, and this may be present at about 1.0% by weight. The recurring meibomian gland disorder may manifest as blepharitis.

In another aspect there is provided a kit for treating recurrent meibomian gland disorder and thereby decreasing the frequency of recurrence of said disorder, said kit comprising a composition comprising about 0.1% by weight dexamethasone and about 1.0% by weight azithromycin in an ophthalmically acceptable sustained release vehicle and instructions for using the composition.

In another aspect there is provided a composition for reducing the frequency of recurrence of meibomian gland disorder in a patient comprising about 0.1% by weight dexamethasone and 1.0% by weight azithromycin in an ophthalmically acceptable vehicle comprising a carboxyl-containing polymer having less than about 5% by weight cross-linking agent.

### DETAILED DESCRIPTION

The present subject matter is directed, in part, to a method of treating recurrent meibomian gland disorder and decreasing the frequency of meibomian gland disorders (MGD) by applying a combination of an antibiotic and a glucocorticoid in a slow release ophthalmic carrier vehicle in the affected eye(s). This combination has been found effective in ameliorating the clinical signs and symptoms associated with meibomian gland disorders and reducing the frequency of recurrence of these disorders. This is in contrast to the standard pharmaceutical intervention which utilizes an antibiotic in combination with an anti-inflammatory agent. Such formulations known in the art are exemplified by TOBRADEX® (0.3% tobramycin and dexamethasone alcohol), CORTISPORIN® (neomycin or polymyxin B (10,000 units) with hydrocortisone), Maxitrol (neomycin or polymyxin B (10,000 units) with dexamethasone), BLEPHAMIDE® (10% sulfacetamide and prednisolone acetate), and VASOCIDIN® (100 mg/mL sulfacetamide & prednisolone sodium phosphate), all of which use relatively high dosage of antibacterial agent. In addition to undesirable side-effects associated with a number of the aforementioned antibiotics, increased concern for the development of drug-resistant bacterial strains provides the impetus for the development of new treatment regimens that move away from using such broad spectrum antibiotics. The need for reduced dependence on these antibiotics for treating meibomian gland disorders such as blepharitis, dry eye and chalazion is met by the present disclosure

Thus, in one embodiment, a method of reducing the frequency of reoccurrence of meibomian gland disorders is provided that includes administering to the eye of a subject an effective amount of active ingredients in an ophthalmically acceptable vehicle. The active ingredient consists essentially of an antibacterial agent and a glucocorticoid, while the ophthalmically acceptable vehicle includes an aqueous polymer suspension that when mixed with tear fluid of the eye provides a sustained release of the active ingredients. The aqueous polymer suspension includes a carboxyl-containing polymer having less than about 5% by weight cross-linking agent and has a viscosity in a range from about 1,000 to about 30,000 centipoises.

As used herein, the term “meibomian gland disorders” includes blepharitis, dry eye, chalazion and all types of ocular diseases characterized by obstruction of the meibomian gland.

As used herein, “administering to the eye of a subject” means administering the active ingredients in an ophthalmically acceptable vehicle in the form of an eye drop directly to the eye and/or in the eyelid margins, such administration techniques being familiar to persons skilled in the art.

As used herein, “an effective amount” when used in connection with treating meibomian gland disorders is intended to qualify the amounts of antibacterial agent and glucocorticoid used in the treatment of MGD and/or prophylaxis against MGD. These amounts will achieve the goal of reducing the reoccurrence or frequency of MGD. An effective amount includes from about 15 to 25 μl in one embodiment and from about 1 to 4 doses in another embodiment an “effective amount” can include a dosage regimen of once per day, twice per day, thrice per day, and so on.

As used herein an “ophthalmically acceptable vehicle” is one which allows delivery of active ingredients to reduce the recurrence of MGD compared to conventional treatments with antibiotics alone or glucocorticoid alone. An ophthalmically acceptable vehicle is one that can maintain proper intraocular pressure and provide solutions that are either isotonic, mildly hypotonic, or mildly hypertonic. To maintain such conditions one can include various non-ionic osmolality-adjusting compounds such as polyhydric alcohols, including for example, glycerol, mannitol, sorbitol, or propylene glycol. Alternatively, osmolality adjusting compounds can include ionic salts such as sodium or potassium chloride. An ophthalmically acceptable vehicle can also include buffers to adjust to an acceptable pH, which can range from about 5.5 to 6.5. Such buffer systems include, for example, acetate buffers, citrate buffers, phosphate buffers, borate buffers and mixtures thereof. Specific useful buffer components include citric acid/sodium citrate, boric acid, sodium borate, sodium phosphates, including mono, di- and tri-basic phosphates, such as sodium phosphate monobasic monohydrate and sodium phosphate dibasic heptahydrate, and mixtures thereof. Any other suitable ophthalmically acceptable buffer components can be employed to maintain the pH of the ophthalmic formulation so that the ophthalmic formulation is provided with an acceptable pH, and the foregoing buffer components are merely exemplary of such buffer components.

As used herein, a “sufficient period” for treatment of blepharitis means a sufficient time to completely resolve clinical signs and symptoms associated with MGD in the eye of a subject and/or reduce re-occurrence of clinical signs and symptoms associated with MGD in the eye of a subject. Such an amount of time can be assessed, for example, by evaluating eradication and/or reduction in the clinical signs or symptoms of MGD and the subject no longer suffers its debilitating effects.

As used herein, “clinical signs or symptoms of blepharitis” include eyelid redness, eyelid swelling, eyelid debris and the clinical symptom of eyelid irritation. Two primary efficacy endpoints used herein to evaluate the efficacy of the combination treatment. Both endpoints are based on the composite score (0-12) of the clinical signs of eyelid redness (0-3), eyelid swelling (0-3), and eyelid debris (0-3), and the clinical symptom of eyelid irritation (0-3).

The first primary efficacy endpoint is “complete clinical resolution” (score of 0) of signs and symptoms after a sufficient time. The second primary efficacy endpoint is recurrence of clinical signs and symptoms by a sufficient time. As used herein “recurrence” is defined as a score of a sufficient period of time after the first primary endpoint (clinical resolution in the study eye) and includes a score of 1 for eyelid redness and a score of 1 for eyelid irritation. As used herein, recurrence is only be evaluated for those who reach clinical resolution at the first primary efficacy endpoint.

As used herein “secondary efficacy variables” include the time to recurrence or exacerbation of clinical signs and symptoms, severity of recurrence or exacerbation, individual signs and symptoms, Investigator's Global Efficacy Rating, and the QOL Questionnaire.

As used herein “an ophthalmically acceptable salt” will include those that exhibit no deleterious effects on the eye as well as being compatible with the active ingredient itself and the components of the ophthalmically acceptable vehicle. Salts or zwitterionic forms of the active ingredient glucocorticoids can be water or oil-soluble or dispersible. The salts can be prepared during the final isolation and purification of the glucocorticoid or separately by adjusting the pH of the appropriate glucocorticoid formulation with a suitable acid or base.

Another aspect provides a method of reducing the reoccurrence of MGD. In some embodiments, an effective amount of an active ingredient is the amount used to reduce the reoccurrence of MGD. These amounts will achieve the goal of reducing the reoccurrence of MGD. An effective amount includes from about 0.1% to 2% per dose of antibiotic and about 0.025% to 0.2% per dose of glucocorticoid. An effective amount includes all values in between and fractions thereof, for example, about 15 μl up to about 50 μl per dose. An effective amount can administered in a dosing regimen once per day, twice per day, thrice per day, or any number of times per day and can be determined in consultation with a physician. An effective amount can be administered as a solution in eye drop form or as a depot as about a 0.05% to about 0.50% by weight solution of the active ingredient, including for example, about 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.25%, 0.30%, 0.35%, 0.40%, 0.45%, and about 0.50% and all values in between and fractions thereof.

In some embodiments, the active ingredients consist essentially of an azalide antibiotic and a glucocorticoid. Azalide antibiotics are a class of macrolide antibiotics that contain a nitrogen in the macrolide ring. The nitrogen imparts unique pharmacokinetic properties and is associated with greater stability of the molecule. One such azalide is the antibiotic azithromycin. Azithromycin (U.S. Pat. No. 4,517,359) is a well-known antibiotic belonging to the macrolide class (of which erythromycin is the precursor). Notwithstanding the structural similarity, azithromycin can be considered as unique within the macrolides class, such as to be included in a new class of antibiotics known as azalides. In particular, the specific characteristics of azithromycin make this molecule more stable, tolerated and effective than its precursor erythromycin (S. Alvarez-Elcoro, M. J. Enzler, “The macrolides: Erythromycin, clarithromycin, and azithromycin”, Mayo Clinic Proceeding, 1999, 74: 613-634).

Azithromycin, even in comparison to other macrolides, shows a superior antibacterial activity against certain gram-negative organisms, while retaining the same efficacy against gram-positive organisms. Moreover azithromycin has an extensive intracellular distribution into specific tissues after oral administration [R. P. Glaude et al., Antimicrob. Agents and Chemother., 1989, 33(3): 277-82]. The extended half-life of azithromycin makes it potentially suitable for once-daily administration against infections of the respiratory tract, skin and soft tissues [A. P. Ball et al., J. Int. Med. Res., 1991, 19(6): 446-50; A. E. Girard et al., Antimicrob. Agents and Chemother., 1987, 31(12): 1948-1954].

Glucocorticoids are potent anti-inflammatory agents and can often be successfully administered independent of the underlying cause of inflammation. Without being bound by theory, glucocorticoids' primary anti-inflammatory mechanism is reported to be related to lipocortin-1 (annexin-1) synthesis. Lipocortin-1 suppresses phospholipase A2, thereby blocking eicosanoid production, and inhibits various leukocyte inflammatory events. In addition, glucocorticoids have been shown to suppress cyclooxygenases, including COX-1 and COX-2.

Glucocorticoids can initiate an anti-inflammatory effect by binding to the cytosolic glucocorticoid receptor (GR). After binding GR, the receptor-ligand complex translocates to the cell nucleus, where it can bind to glucocorticoid response elements (GRE) in the promoter region of target genes. The proteins encoded by these up regulated genes have a wide range of effects including anti-inflammatory effects mediated, for example, by lipocortin I as described above. Glucocorticoids can also reduce the transcription of pro-inflammatory genes by a mechanism of transrepression. Thus, inflammation associated with blepharitis can be ameliorated by glucocorticoid treatment.

In some embodiments, the active ingredients are azithromycin and a glucocorticoid including, for example, hydrocortisone, cortisone acetate, prednisone, prednisolone, methylprednisolone, dexamethasone, betamethasone, triamcinolone, beclomethasone, Fluorometholone, and combinations thereof. Other glucocorticoids useful in the method for treating blepharitis include, for example, 21-acetoxypregnenolone, alclometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone, prednisolone 25-diethylamino-acetate, prednisolone sodium phosphate, prednival, prednylidene, rimexolone, tixocortol, triamcinolone acetonide, triamcinolone benetonide, triamcinolone hexacetonide, their opthalmically acceptable salts, combinations thereof, and mixtures thereof. In one embodiment, the glucocorticoid includes dexamethasone, prednisone, prednisolone, methylprednisolone, medrysone, triamcinolone, loteprednol etabonate, opthalmically acceptable salts thereof, combinations thereof, and mixtures thereof.

The effects of treating MGD with azithromycin and dexamethasone, in particular, with the aid of the slow-release ophthalmically acceptable carrier, are shown in the Example below. In accordance with various embodiments, dexamethasone includes, for example, dexamethasone sodium phosphate, dexamethasone (alcohol), dexamethasone acetate, dexamethasone dimethylbutyrate, dexamethasone trimethylacetate, dexamethasone dipropionate, dexamethasone acefurate, and mixtures thereof.

In some embodiments, the ophthalmically acceptable vehicle uses insoluble polymers to provide a gel or liquid drops which release the drug over time. The polymer is about 0.1 to about 6.5% in some embodiments, and, in other embodiments about 1.0 to about 1.3% by weight based on the total weight of the suspension of a cross-linked carboxy-containing polymer. Suitable carboxy-containing polymers are described, for example, in U.S. Pat. No. 5,192,535 to Davis et al. which is hereby incorporated by reference. These polymer carriers include lightly crosslinked carboxy-containing polymers (such as polycarbophil, or CARBOPOLS®), dextran, cellulose derivatives, polyethylene glycol 400 and other polymeric demulcents such as polyvinylpyrolidone, polysaccaride gels and GELRITE®. In some embodiments, the ophthalmically acceptable vehicle is carboxy-containing polymer system known by the trade name DuraSite®. Durasite is a sustained release topical ophthalmic delivery system that releases the drug at a controlled rate.

In accordance with some embodiments, a sustained release topical ophthalmically acceptable carrier includes an aqueous suspension at a pH of from about 3 to about 6.5 and an osmotic pressure of from about 10 to about 400 mOsM containing from about 0.1% to about 6.5% by weight, based on the total weight of the suspension, of a carboxyl-containing polymer prepared by polymerizing one or more carboxyl-containing monoethylenically unsaturated monomers and less than about 5% by weight of a cross-linking agent, such weight percentages of monomers being based on the total weight of monomers polymerized. The suspension has an initial viscosity of from about 1,000 to about 30,000 centipoises and is administrable to the eye in drop form at that initial viscosity. The polymer has average particle size of not more than about 50 μm, preferably not more than about 30 μm, in equivalent spherical diameter. It is lightly cross-linked to a degree such that although the suspension is administrable in drop form, upon contact of the lower pH suspension with the higher pH tear fluid of the eye, the suspension is rapidly gellable to a substantially greater viscosity than the viscosity of the suspension as originally administered in drop form. Accordingly, the resulting more viscous gel can remain in the eye for a prolonged period of time so as to release a medicament contained therein in sustained fashion.

The polymer is, in one embodiment, prepared from at least about 50% by weight, and in other embodiments from at least about 90% by weight, of one or more carboxyl-containing monoethylenically unsaturated monomers. The polymer can be prepared by suspension or emulsion polymerizing acrylic acid and a non-polyalkenyl polyether difunctional cross-linking agent to a particle size of not more than about 50 μm in one embodiment, and not more than about 30 μm, in equivalent spherical diameter, in other embodiments. In one embodiment, the cross-linking agent is divinyl glycol. In other embodiments, one can replace up to about 40% by weight of the carboxyl-containing monoethylenically unsaturated monomers by one or more non-carboxyl-containing monoethylenically unsaturated monomers containing only physiologically and ophthamologically innocuous substituents.

The osmotic pressure is, in some embodiments, achieved by using a physiologically and ophthalmologically acceptable salt in an amount of from about 0.01% to about 1% by weight, based on the total weight of the suspensions. Exemplary salts include potassium and sodium chlorides.

In some embodiments, the foregoing suspensions are prepared and packaged at the desired viscosity of from 1,000 to about 30,000 centipoises for administration to the eye in drop form. In one exemplary drug delivery method, the foregoing suspensions, containing the active ingredient(s), are administered to the eye at an initial viscosity in drop form to cause the administered suspension. Upon contact with the higher pH tear fluid of the eye the suspension rapidly gels in situ to a substantially greater viscosity. This more viscous gel remains in the eye for a prolonged period of time so as to release the active ingredient(s), entrapped in the gel that forms in situ, in sustained fashion.

The aqueous suspensions can contain amounts of lightly cross-linked polymer particles ranging from about 0.1% to about 6.5% by weight, and in other embodiments from about 0.5% to about 4.5% by weight, based on the total weight of the aqueous suspension. They can be prepared using pure, sterile water, preferably deionized or distilled, having no physiologically or ophthalmologically harmful constituents, and will be adjusted to a pH of from about 3.0 to about 6.5, and in other embodiments from about 4.0 to about 6.0, using any physiologically and ophthalmologically acceptable pH adjusting acids, bases or buffers, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylaminomethane), or the like and salts and buffers such as citrate/dextrose, sodium bicarbonate, ammonium chloride and mixtures of the aforementioned acids and bases.

When formulating the aqueous suspensions, their osmotic pressure will be adjusted to from about 10 milliosmolar (mOsM) to about 400 mOsM, and preferably from about 100 to about 250 mOsM, using appropriate amounts of physiologically and ophthalmologically acceptable salts. Sodium chloride can be used to approximate physiologic fluid, and amounts of sodium chloride ranging from about 0.01% to about 1% by weight, and in other embodiments from about 0.05% to about 0.45% by weight, based on the total weight of the aqueous suspension, will give osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfite and the like, e.g., potassium chloride, sodium thiosulfate, sodium bisulfite, ammonium sulfate, and the like can also be used in addition to or instead of sodium chloride to achieve osmolalities within the above-stated ranges.

The amounts of lightly cross-linked polymer particles, the pH, and the osmotic pressure chosen from within the above-stated ranges can be correlated with each other and with the degree of cross-linking to give aqueous suspensions having viscosities ranging from about 1,000 to about 30,000 centipoise, and in other embodiments from about 5,000 to about 20,000 centipoise, as measured at room temperature (about 25° C.) using a Brookfield Digital LVT Viscometer equipped with a number 25 spindle and a 13R small sample adapter at 12 rpm. The correlations of those parameters are also such that the suspensions will gel on contact with tear fluid to give gels having viscosities estimated to range from about 30,000 to about 100,000 centipoise, e.g., from about 200,000 to about 300,000 centipoise, measured as above, depending on pH as observed, for example, from pH-viscosity curves. This effect is noted by observing a more viscous drop on the eye as a set cast. The cast, after setting, can be easily removed.

The viscous gels that result from fluid eye drops delivered by means of the aqueous suspensions have residence times in the eye ranging from about 2 to about 12 hours, e.g., from about 3 to about 6 hours. The active ingredients contained in these ophthalmically acceptable vehicles can be released from the gels at rates that depend on such factors as the active ingredient itself and its physical form, the extent of drug loading and the pH of the system, as well as on any drug delivery adjuvants, such as ion exchange resins compatible with the ocular surface, which can also be present. For fluorometholone, for example, release rates in the rabbit eye in excess of four hours, as measured by fluorometholone contained in the aqueous humor, have been observed.

The compositions can be formulated in any of several ways. For example the active ingredient(s), the lightly cross-linked polymer particles, and the osmolality-adjusting salt can be preblended in dry form, added to all or part of the water, and stirred vigorously until apparent polymer dispersion is complete, as evidenced by the absence of visible polymer aggregates. Sufficient pH adjusting agent is then added incrementally to reach the desired pH, and more water to reach 100 percent formula weight can be added at this time, if necessary. Another convenient method involves adding the drug to about 95 percent of the final water volume and stirring for a sufficient time to saturate the solution. Solution saturation can be determined in known manner, e.g., using a spectrophotometer. The lightly cross-linked polymer particles and the osmolality-adjusting salt are first blended in dry form and then added to the drug-saturated suspension and stirred until apparent polymer hydration is complete. Following the incremental addition of sufficient pH adjusting agent to reach the desired pH, the remainder of the water is added, with stirring, to bring the suspension to 100 percent formula weight.

These aqueous suspensions can be packaged in preservative-free, single-dose non-reclosable containers. This permits a single dose of the active ingredient to be delivered to the eye one drop at a time, with the container then being discarded after use. Such containers eliminate the potential for preservative-related irritation and sensitization of the corneal epithelium, as has been observed to occur particularly from ophthalmic medicaments containing mercurial preservatives. Multiple-dose containers can also be used, if desired, particularly since the relatively low viscosities of the aqueous suspensions permit constant, accurate dosages to be administered dropwise to the eye as many times each day as necessary.

In those vehicles where preservatives are to be included, suitable preservatives are chlorobutanol, Polyquat, benzalkonium chloride, cetyl bromide, benzethonium chloride, cetyl pyridinium chloride, benzyl bromide, EDTA, phenylmercury nitrate, phenylmercury acetate, thimerosal, merthiolate, acetate and phenylmercury borate, chlorhexidine, polymyxin B sulphate, methyl and propyl parabens, phenylethyl alcohol, quaternary ammonium chloride, sodium benzoate, sodium proprionate, sorbic acid, and sodium perborate. In particular embodiments, the preservative includes benzalkonium chloride.

In some embodiments, the preservative is present in a range from about 0.001 to about 0.005% by weight. The preservative can be present at about 0.001, 0.002, 0.003, 0.004, 0.005 and any amount in between these amounts. In particular, the present subject matter has the benefit of substantial reduction in the use of a bactericidal component. Thus, in some embodiments, the present disclosure provides an ophthalmically acceptable vehicle having less than about 0.01% of a preservative with bactericidal activity in one embodiment, and less than about 0.009%, 0.008%, 0.007%, 0.006%, 0.005%, 0.004%, 0.003%, 0.002%, or 0.001%, in other embodiments.

In some embodiments, the ophthalmically acceptable vehicle includes a wetting agent. Such agents can be useful in distributing the active ingredient in an otherwise predominantly aqueous environment. Such wetting agents include, for example, Poloxamer 407, a triblock copolymer consisting of a central hydrophobic block of polypropylene glycol flanked by two hydrophilic blocks of polyethylene glycol. Other wetting agents that can be used include carboxymethylcellulose, hydroxypropyl methylcellulose, glycerin, mannitol, polyvinyl alcohol and hydroxyethylcellulose.

In some embodiments, the ophthalmically acceptable vehicle can include a thickening agent or viscosfier that modulates the viscosity of the vehicle. These include, without limitation, polyvinyl alcohol, polyacrylic acid, polyethylene oxide, and chitosan.

In some embodiments, the present subject matter is directed to a kit which includes: (a) a composition comprising about 0.1% by weight dexamethasone and 1.0% by weight azithromycin in an ophthalmically acceptable capable of slow release as detailed herein and (b) instructions for using the composition of (a) for treating recurrent meibomian gland disorders and thereby decreasing the frequency of recurrence.

In some embodiments, the kit further includes a means for administering the composition. In some embodiments, the means for administering can include a bottle, dropper, cup, specialized eye-wash apparatus, wetted towel or sponge. In some embodiments, the kit includes a cleaning apparatus (e.g., a towel, pad, cloth, brush, sponge, etc.) and/or a cleaning solution (e.g., purified water, a detergent solution, a boric acid solution, etc.). In some embodiments of the present disclosure, the ocular area is cleaned prior to administration of the composition of the present subject matter.

The composition can be packaged for a single dose administration, e.g., in a bottle, jar, ampoule, tube, syringe, envelope, container, vial or the like. When the composition is individually packaged, in some embodiments, the composition does not include a preservative. Alternatively, the composition can be contained in a package that is capable of holding multiple units; e.g., in resealable glass or plastic packages. In some kits, the components of the composition are mixed together immediately preceding their usage. For example, in some embodiments one or more dry components of the composition of the kit are packaged in a separate container; e.g., a plastic bottle, and then mixed with one or more of the liquid components of the composition immediately prior to use. Optionally, the kit can include a dropper or other device for transferring or administering the composition to a subject.

The kit can further include instructions for using the composition. For example, such instructions can be in a form prescribed by a governmental agency regulating the manufacture, use or sale of pharmaceuticals or biological products, which reflects approval by the agency of the manufacture, use or sale for human application. In some embodiments, the kit further includes information on the use of the composition or a pre-recorded media device which, e.g., provides information on the use of the method.

The kit can also include a container for storing the components of the kit. The container can be, for example, a bag, box, envelope or any other container suitable for use. In some embodiments, the container is large enough to accommodate each component. However, in some cases, it can be desirable to have a smaller container which is large enough to carry only some of the components.

It is understood that modifications which do not substantially affect the activity of the various embodiments are also included within the definition provided herein. Accordingly, the following examples are intended to illustrate but not limit.

### EXAMPLE

The following Example shows a composition with 1.0% Azithromycin and 0.1% Dexamethasone that is useful in a method for treating recurrent MGD.

Table 1 below provides an exemplary formulation of azithromycin 1.0% and glucocorticoid dexamethasone as 0.1% in an exemplary ophthalmically acceptable vehicle.

TABLE 1

| INGREDIENT | CONCENTRATION (% W/W) |
|---|---|
| Azithromycin | 1.0 |
| Dexamethasone, USP | 0.10 |
| Mannitol, USP | 1.0 |
| Citric Acid Anhydrous, USP | 0.20 |
| Sodium Citrate Dihydrate, | 0.14 |
| USP | |
| Poloxamer 407, NF | 0.20 |
| Benzalkonium Chloride, NF | 0.003 |
| Polycarbophil, USP | 0.90 |
| Sodium Chloride, USP | 0.45 |
| Edetate Disodium Dihydrate, | 0.10 |
| USP | |
| Sodium Hydroxide, 2N | Adjust to pH 6.3 |
| Water For Injection, USP | q.s. to 100% |

In this Example, subjects with recurrence of clinically-diagnosed blepharitis who had previously been treated for the meibomian gland disorder with a therapy other than a combination of azithromycin and dexamethasone or who had not received treatment for the initial occurrence will be treated with either the azithromycin and dexamethasone formulation in the vehicle study; azithromycin alone in the vehicle, dexamethasone alone in the vehicle and the vehicle alone.

Informed consent, demographic information, and initial examination of the eyes will be performed, and subjects having a minimum combined score of 5 as described above for the following signs and/or symptoms in at least one eye: Eyelid redness, Eyelid swelling, Eyelid debris, and Eyelid irritation will be selected. In addition, the eye will be required to have a minimum score of 1 for eyelid redness, and a minimum score of 1 for eyelid irritation.

Following the selection for subjects, for 14 days, some selected subjects with clinically-diagnosed recurrence of blepharitis will be treated with either the azithromycin and dexamethasone formulation in the vehicle; azithromycin alone in the vehicle, dexamethasone alone in the vehicle or the vehicle alone. Subjects that do not reach clinical resolution (score of 0) or improvement in signs and symptoms at Day 15 will exit the study.

Subjects will be evaluated following day 15 for any reoccurrence of symptoms of MGD including eyelid redness, eyelid swelling, eyelid debris, and eyelid irritation. Recurrence as used herein is a score of from Day 15 in the study eye and includes a score of 1 for eyelid redness and a score of 1 for eyelid irritation. Recurrence will only be evaluated for those who reach clinical resolution at Day 15.

All efficacy analyses will be conducted on the Intent-to-Treat population, defined as all subjects randomized. Additional efficacy analyses may be conducted on the Per Protocol population, defined as all subjects that received at least one dose of IMP with no significant protocol deviations. The Safety population, defined as all subjects that received at least one dose of IMP, will be used for all safety analyses. Primary and secondary efficacy analyses will use data from the study eye only. Additional analyses may examine data from the fellow eye. Safety analyses will include data from the treated eye(s).

The first primary efficacy analysis compares the incidence of clinical resolution of signs and symptoms at day 15 between the combination azithromycin and Dexamethasone in vehicle formulation and the Azithromycin alone in the groups. The second primary efficacy analysis compares the incidence of recurrence of clinical signs and symptoms by 6-Month Follow-up between the azithromycin and dexamethasone alone in vehicle formulation and dexamethasone alone in vehicle groups. The second primary efficacy analysis is performed only if dexamethasone was demonstrated to be superior to vehicle for the endpoint of clinical resolution of signs and symptoms at Day 15. Two-sided, chi-square tests with alpha of 0.05 will be used for both comparisons. Fisher's exact test, or Cochran-Mantel-Haenszel test, using investigation site as strata, may replace the chi-square test if appropriate. No adjustments were made for multiple comparisons. Missing data was be imputed using the Last Observation Carried Forward (LOCF) method.

Additional efficacy analyses include the time to recurrence or exacerbation of clinical signs and symptoms. Comparisons between groups will be made using the Log-Rank Test from the Kaplan-Meier analysis. The treatment groups may also be compared for the severity of recurrence or exacerbation of clinical signs and symptoms. This will be performed with a t-test. Comparisons between the treatment groups will also be made for the Investigator's Global Efficacy Rating throughout the study. Wilcoxon Rank Sum tests will be used to test the comparisons at various time points. The analysis of “change from baseline” for individual signs and symptoms will be performed with a t-test. The incidence, timing, and severity of exacerbation (increase ≧4) of clinical signs and symptoms for subjects with scores greater than 0 at Day 15 may also be analyzed. A comparison between ISV-502 and vehicle and a comparison between 0.1% Dexamethasone and vehicle may also be performed.

Sensitivity analyses among Per Protocol subjects will be performed to demonstrate the robustness of the analysis results. Additional sensitivity analyses may be performed to use alternate methods to impute missing data, such as Baseline Observation Carried Forward (BOCF) or subjects with missing values classified as failure.

Throughout this application various publications have been referenced within parentheses. The disclosures of these publications in their entireties are hereby incorporated by reference in this application in order to more fully describe the state of the pertinent art.

Although the present subject matter has been described with reference to the disclosed embodiments, those skilled in the art will readily appreciate that the specific examples and studies detailed above are only illustrative. It should be understood that various modifications can be made without departing from the spirit of the present subject matter. Accordingly, the invention is limited only by the following claims.

1. Use of a composition comprising a therapeutically effective amount of an azalide antibiotic and a glucocorticoid for the treatment of recurring meibomian gland disorder in a patient, wherein said composition reduces the frequency of recurrence of the meibomian gland disorder.

2. The use of the composition of claim 1 wherein previous occurrence or occurrences of meibomian gland disorder were treated with a therapy other than a combination of azalide antibiotic and a glucocorticoid.

3. The use of the composition of claim 1 wherein the composition further comprises an ophthalmically acceptable aqueous polymer suspension vehicle comprising a carboxyl-containing polymer having less than about 5% by weight cross-linking agent.

4. The use of the composition of claim 1 wherein the glucocorticoid is dexamethasone.

5. The use of the composition of claim 4 wherein the dexamethasone is present at about 0.1% by weight.

6. The use of the composition of claim 1 wherein the azalide antibiotic is azithromycin.

7. The use of the composition of claim 6 wherein the azithromycin is present at about 1.0% by weight.

8. The use of the composition of claim 1 wherein the recurring meibomian gland disorder manifests as blepharitis.

9. The use of an azalide antibiotic and a glucocorticoid for the manufacture of a composition for the treatment of recurring meibomian gland disorder in a patient, wherein said composition reduces the frequency of recurrence of the meibomian gland disorder.

10. The use of the azalide antibiotic and the glucocorticoid in accordance with claim 9 wherein previous occurrence or occurrences of meibomian gland disorder were treated with a therapy other than a combination of azalide antibiotic and a glucocorticoid.

11. The use of the azalide antibiotic and the glucocorticoid in accordance with claim 9 wherein the composition further comprises an ophthalmically acceptable aqueous polymer suspension vehicle comprising a carboxyl-containing polymer having less than about 5% by weight cross-linking agent.

12. The use of the azalide antibiotic and the glucocorticoid in accordance with claim 9 wherein the glucocorticoid is dexamethasone.

13. The use of the azalide antibiotic and the glucocorticoid in accordance with claim 12 wherein the dexamethasone is present at about 0.1% by weight.

14. The use of the azalide antibiotic and the glucocorticoid in accordance with claim 9 wherein the azalide antibiotic is azithromycin.

15. The use of the azalide antibiotic and the glucocorticoid in accordance with claim 14 wherein the azithromycin is present at about 1.0% by weight.

16. The use of the azalide antibiotic and the glucocorticoid in accordance with claim 9 wherein the recurring meibomian gland disorder manifests as blepharitis.

17. A method of treating a recurring meibomian gland disorder in a patient, comprising administering to a patient suffering from recurring meibomian gland disorder a composition comprising a therapeutically effective amount of an azalide antibiotic and a glucocorticoid, thereby reducing the frequency of recurrence of the meibomian gland disorder.

18. The method of claim 17 wherein previous occurrence or occurrences of meibomian gland disorder were treated with a therapy other than a combination of azalide antibiotic and a glucocorticoid.

19. The method of claim 17 wherein the composition further comprises an ophthalmically acceptable aqueous polymer suspension vehicle comprising a carboxyl-containing polymer having less than about 5% by weight cross-linking agent

20. The method of claim 17 wherein the glucocorticoid is dexamethasone.

21. The method of claim 20 wherein the dexamethasone is present at about 0.1% by weight.

22. The method of claim 17 wherein the azalide antibiotic is azithromycin.

23. The method of claim 22 wherein the azithromycin is present at about 1.0% by weight.

24. The method of claim 17 wherein the recurring meibomian gland disorder manifests as blepharitis.

25. A kit for treating recurrent meibomian gland disorder and thereby decreasing the frequency of recurrence of said disorder, said kit comprising a composition comprising about 0.1% by weight dexamethasone and about 1.0% by weight azithromycin in an ophthalmically acceptable sustained release vehicle and instructions for using the composition.

26. A composition for reducing the frequency of recurrence of meibomian gland disorder in a patient comprising about 0.1% by weight dexamethasone and 1.0% by weight azithromycin in an ophthalmically acceptable vehicle comprising a carboxyl-containing polymer having less than about 5% by weight cross-linking agent.
